(19) United States
(12) Patent Application Publication
(10) Pub. No.: US 2022/0304947 A1
(43) Pub. Date: Sep. 29, 2022

(54) COMPOSITIONS AND METHODS FOR INHIBITING PROTEOLYTIC ACTIVATION OF VIRUSES

(71) Applicants: Muhammed Majeed, Bangalore (IN); Kalyanam Nagabhushanam, East Windsor, NJ (US); Lakshmi Mundkur, Bangalore (IN)

(72) Inventors: Muhammed Majeed, Bangalore (IN); Kalyanam Nagabhushanam, East Windsor, NJ (US); Lakshmi Mundkur, Bangalore (IN)

(21) Appl. No.: 17/500,479

(22) Filed: Oct. 13, 2021

Related U.S. Application Data
(60) Provisional application No. 63/165,808, filed on Mar. 25, 2021.

(51) Int. Cl. A61K 31/12; A61P 31/14

(52) U.S. Cl. CPC A61K 31/12; A61P 31/14

(57) ABSTRACT

The present invention discloses use of a composition comprising enriched in bisdemethoxycurcumin, specifically a composition comprising not less than 20% w/w bisdemethoxycurcumin, 10-35% w/w demethoxycurcumin and 10-50% curcumin in preventing the activation of viral spike proteins by inhibiting the activity of proteolytic enzymes and in inhibiting the replication and growth of pathogenic viruses, specifically SARS-CoV-2 in mammalian cells. The invention also discloses the potential of the above composition in managing SARS-CoV-2 induced infections.

### CROSS-REFERENCE TO RELATED APPLICATIONS

This is a non-provisional US patent application claiming priority from U.S. Provisional application 63/165,808, filed on 25 Mar. 2021, the details of which are being incorporated herein by reference.

### FIELD OF INVENTION

The invention in general relates to compositions for preventing viral infections. Specifically, the invention discloses the anti-viral potential of composition comprising bisdemethoxycurcumin. Still more specifically, the invention discloses the anti-viral potential of a curcuminoid composition comprising 20-80% w/w bisdemethoxycurcumin, 10-35% w/w demethoxycurcumin and 10-50% w/w curcumin by inhibiting the proteolytic activation of viruses.

### BACKGROUND OF INVENTION

Viruses are the most common agent of respiratory diseases in mammals. Respiratory viruses that most commonly cause infections belong to the class of influenza virus, respiratory syncytial virus, parainfluenza viruses, metapneumovirus, rhinovirus, coronaviruses, adenoviruses, and bocaviruses (Boncristiani et al., Respiratory Viruses. Encyclopedia of Microbiology. 2009: 500-518). The world has seen many endemics and pandemics caused by these respiratory virus, notable ones include Spanish flu, H1N1 endemic, Swine flu, MERS-CoV, Sars-Co V-1 and Sars-CoV-2. The coronavirus disease COVID-19 caused by Sars-CoV-2 caused the worst pandemic the world has ever seen in more than a century. Reports indicate the potential of further pandemics arising due to zoonotic transmission from animals (Lee et al., Emerging threats from zoonotic coronaviruses—from SARS and MERS to 2019-CoV, Journal of Microbiology, Immunology and Infection, Volume 53, Issue 3, June 2020, Pages 365-367). The threats of using these virus as a tool for biowarfare also looms around.

The mechanism of infection of SARS-CoV-2 consists of the below mentioned steps: (Andrew G. Harrison et al., Mechanisms of SARS-CoV-2 Transmission and Pathogenesis, Trends in Immunology, 2020).
1. Binding of Spike protein to ACE2 on human cells
2. Entry of viral particles into the cells
3. Fusion of the Endosome—Lysosome and release of viral RNA
4. Translation of viral RNA using the host RNA polymerase
5. Viral replication
6. Transcription and translation of structural proteins
7. Assembly and release of viral particles

The binding of spike protein of the virus to the ACE 2 receptor on human cells is the first step in the viral pathogenesis which facilitates the entry of the virus into the cells. The surface spike is composed of S1 subunit, which harbors the receptor binding domain (RBD), and the S2 subunit, which anchors the spike to the viral envelope, following protease activation. The Spike glycoprotein of CoV-2 harbors a Furin cleavage site, at the boundary between its two subunits, S1/S2. The host cell enzyme Furin proprotein convertase cleaves the spike protein at this site to make the virus more susceptible to its primary receptor, angiotensin-converting enzyme-2 or ACE2. Upon binding of the S protein to ACE2, the S protein goes through a conformational change that exposes a cleavage site between the S1 and the S2 domains. S1 then dissociates from S2 and the truncated 2 subunit of the Spike protein facilitates fusion of viral and cellular membranes.

Proteolytic activation of the S protein by host proteases at the putative cleavage site located at the S1/S2 boundary and within the S2 domain (ST cleavage site) has been documented to be critical for its fusion activity in coronaviruses (CoVs). Many proteases have been found to activate CoVs in vitro, including Furin, cathepsin L, and trypsin-like serine proteases such as the transmembrane serine protease 2 (TMPRSS2). Furin cleaves the precursors of a broad range of proteins, including hormones, growth factors, cell surface receptors, and adhesion molecules at multibasic motifs of the preferred consensus sequence R-X-R/K-R↓. TMPRSS2 is also widely expressed in epithelial cells of the respiratory, gastrointestinal, and urogenital tract and cleaves at single arginine or lysine residues R/K↓ (Giovanni A. Rossi et al., Differences and similarities between SARS-CoV and SARS-CoV-2: spike receptor-binding domain recognition and host cell infection with support of cellular serine proteases, Infection, 2020).

Generally, some proteins are inactive when they are first synthesized, due to the presence of unwanted sections. These sections must be proteolytically removed in order to become active. There are many proteolytic enzymes which cleave the unwanted sections and activates the proteins. One such proteolytic enzyme is Furin, encoded by the FURIN gene. The importance of Furin and other proteolytic enzymes is provided in the below prior art documents:
1. Wise R J et al., “Expression of a human proprotein processing enzyme: correct cleavage of the von Willebrand factor precursor at a paired basic amino acid site”. Proceedings of the National Academy of Sciences of the United States of America, 1990.
2. Kiefer M C et al., “Identification of a second human subtilisin-like protease gene in the fes/fps region of chromosome 15”. DNA and Cell Biology, 1991.
3. Elisabeth Braun & Daniel Sauter, Furin-mediated protein processing in infectious diseases and cancer Clinical & Translational Immunology 2019; e1073. doi: 0.1002/cti2.1073.
4. Lin et al., Soluble hemojuvelin is released by proprotein convertase-mediated cleavage at a conserved polybasic RNRR site, Blood Cells, Molecules & Diseases, Volume 40, Issue 1, 2008, Pages 122-131
5. Kuninger et al., Pro-protein convertases control the maturation and processing of the iron-regulatory protein, RGMc/hemojuvelin, BMC Biochem 9, 9 (2008). https://doi.org10.1186/1471-2091-9-9

Furin is involved in tumor development and progression, activation of bacterial exotoxins, viral protein processing and pathogenicity, Activation of highly pathogenic viruses which include Corona Virus, Human immunodeficiency viruses (HIV), Highly pathogenic avian influenza A viruses, Ebola and Marburg viruses, Flaviviruses, Papillomaviruses and Hepatitis B virus.

Evidence indicate that SARS-CoV-2 has acquired a Furin cleavage site in the S1-S2 junction (Canrong et al., Furin: A Potential Therapeutic Target for COVID-19, iScience. 2020 Oct. 23; 23(10):101642). Development of specific antiviral agents is an urgent unmet need for SARS-coronavirus 2 (SARS-CoV-2) infection. Furin inhibitors show antiviral effects on SARS-CoV-2-infected cells by decreasing virus production and cytopathic effects. Therefore, Furin inhibitors may be promising antiviral agents for prevention and treatment of variety of viral infection including SARS-CoV-2 infection.

Currently, scientists all around the world are tirelessly working on finding a suitable drug moiety to manage the infections caused by viruses and to enhance the immunity. Natural molecules, with anti-viral potential are also being tested to inhibit the proteolytic activation of viruses. The following prior art documents disclose the potential of different natural molecules on the inhibiting virus, specifically SARS-CoV-2:
1. Sharma et al., IN 202011034071—A COMPOSITION FOR ANTI-CORONA VIRUS APPLICATION,
2. Li et al., Identification of natural compounds with antiviral activities against SARS-associated coronavirus, Antiviral Res. 2005; 67(1): 18-23.
3. Islam et al., Natural products and their derivatives against coronavirus: A review of the non-clinical and pre-clinical data, Phytotherapy Research. 2020; 34:2471-2492.
4. Verma et al., Anti-SARS-CoV Natural Products With the Potential to Inhibit SARS-CoV-2 (COVID-19), Frontiers in Pharmacology, 2020; 11:1-20.

Turmeric extracts comprising Curcumin are reported to be effective in ameliorating the effect of virus (Manoharan et al., Curcumin: A Wonder Drug as a Preventive Measure for COVID19 Management, Indian J Clin Biochem. 2020; 35(3): 373-375). Turmeric has different compounds of which curcumin, demethoxycurcumin and bisdemethoxycurcumin (collectively call curcuminoids) are the major players. The biological properties of curcumin, bisdemethoxycurcumin and demethoxycurcumin vary in different diseases conditions and recently bisdemethoxycurcumin and demethoxycurcumin are garnering the much attention owing to their similar and superior efficacy over curcumin in managing certain disease conditions. (Majeed et al., Reductive Metabolites of Curcuminoids, Nutriscience Publishers LLC, 2019). The anti-viral effects of bisdemethoxycurcumin are reported in the following prior art documents based on in-silico experimental results:
1. Gandhi et al., IN 202111010632—A NOVEL DRUG FOR SARS-COV-2
2. Sharma et al., In-silico screening of plant-derived antivirals against main protease, 3CLpro and endoribonuclease, NSP15 proteins of SARS-CoV-2, Journal of Biomolecular Structure and Dynamics, 2020.DOI: 10.1080/07391102.2020.1808077.
3. Patel et al., Virtual screening of curcumin and its analogs against the spike surface glycoprotein of SARS-CoV-2 and SARS-CoV, Journal of Biomolecular Structure and Dynamics, 2021. DOI: 10.1080/07391102.2020.1868338

However, there are limitations in the study design of in-silico experiments and docking studies are generally not consistent with ligand-based studies (Chen, Yu-Chian, Beware of docking!, Trends in Pharmacological Sciences, 2015, 36(2):78-95). Docking studies do not always account for protein flexibility although huge conformational space is available to protein structures. Further, the models do not take the water molecules into consideration which in protein-ligand interaction is ubiquitous. Because of its small size and a surface that is composed entirely of hydrogen bond donors and acceptors, it is supramolecularly very potent in determining the binding interactions and calculation of energies. Further, a lack of accurate scoring function, lack of a synergistic computer model for multi-drug effect assessment and multi domain proteins, lack of standardization for testing and validating results also affect the efficacy and reliability of docking studies. Further, docking studies cannot predict if a ligand is an agonist or an antagonist based on the binding energies (Ray A, 7 Limitations of Molecular Docking & Computer Aided Drug Design and Discovery. www.amitray.com. 2018 Oct. 21 Available at: https://amitray.com/7-limitations-of-molecular-docking-computer-aided-drug-design-and-discovery/[Accessed 25 Aug. 2021]. The methods also do not take into account the mutation rate which is more prevalent in virus. Thus, in-silica results are not reliable in predicting the potential of curcuminoids, specifically bisdemethoxycurcumin in inhibiting viral replication.

Furthermore, the current drugs used for the treatment of SARS-CoV-2 infections elicit their effect by inhibiting glycosylation of host receptors, proteolytic processing, endosomal acidification, inhibition of 3-chymotrypsin-like protease, inhibition of integrase strand transfer, inhibition of viral RNA-dependent RNA polymerase, or ACE-2 inhibition. A person skilled in the art would ascertain that all these targets are unique and distinct. The present invention discloses the anti-viral potential of a curcuminoid composition comprising not less than 20% w/w bisdemethoxycurcumin by inhibiting the proteolytic activation of viral spike protein which is non-obvious and industrially viable.

It is the principal object of the invention to disclose the potential of a composition comprising not less than 20% w/w bisdemethoxycurcumin in preventing the activation of viral spike proteins by inhibiting the activity of proteolytic enzymes.

It is another object of the invention to disclose the potential of a composition comprising not less than 20% w/w bisdemethoxycurcumin in inhibiting the replication of pathogenic viruses, specifically SARS-CoV-2.

It is yet another object of the invention to disclose the use of a composition comprising not less than 20% w/w bisdemethoxycurcumin in managing infections caused by pathogenic viruses, specifically SARS-CoV-2 in mammals.

The present invention satisfies the above mentioned objectives and provides further related advantages.

### SUMMARY OF THE INVENTION

In a most preferred embodiment, the invention discloses use of a curcuminoid composition comprising not less than 20% w/w bisdemethoxycurcumin, 10-35% w/w demethoxycurcumin and 10-50% w/w curcumin in preventing the activation of viral spike proteins by inhibiting the activity of proteolytic enzymes.

In another preferred embodiment, the invention discloses use a composition comprising not less than 20% w/w bisdemethoxycurcumin, 10-35% w/w demethoxycurcumin and 10-50% w/w curcumin in inhibiting the replication and growth of pathogenic viruses in mammalian cells.

In another most preferred embodiment, the invention discloses use of a composition comprising not less than 20% w/w bisdemethoxycurcumin, 10-35% w/w demethoxycurcumin and 10-50% w/w curcumin for managing infections caused by viruses, specifically SARS-CoV-2 infections in mammals.

Other features and advantages of the present invention will become apparent from the following more detailed description, taken in conjunction with the accompanying images, which illustrate, by way of example, the principle of the invention.

### BRIEF DESCRIPTION OF DRAWINGS

FIG. 1 is a graphical representation showing the IC₅₀ values of a composition comprising 20% w/w bisdemethoxycurcumin, 10-35% w/w demethoxycurcumin and 10-50% w/w curcumin (AC3 complex) compared to 95% w/w curcumin, 95% w/w bisdemethoxycurcumin (BDMC) and Curcumin enriched complex (C3 complex) in inhibiting the activity of the enzyme Furin.

### DESCRIPTION OF PREFERRED EMBODIMENTS

In a most preferred embodiment, the invention discloses a method of preventing the activation of viral spike proteins by inhibiting the activity of proteolytic enzymes, said method comprising step of bringing into contact proteolytic enzymes with a composition comprising not less than 20% w/w bisdemethoxycurcumin, to bring about an effect of inhibiting the activity of the enzyme. In a related aspect, the composition comprises of 20-80% w/w bisdemethoxycurcumin. In a further related aspect, the composition comprises of 30-70% bisdemethoxycurcumin. In a further aspect, the composition comprises 40-60% bisdemethoxycurcumin. In a related aspect, the composition further comprises 10-35% w/w demethoxycurcumin and 10-50% w/w curcumin. In a related aspect, the composition further comprises 10-35% w/w demethoxycurcumin and 10-45% w/w curcumin. In another related aspect, the composition comprises of 30-50% w/w bisdemethoxycurcumin, 10-25% w/w demethoxycurcumin and 30-50% w/w curcumin. In another related aspect, the composition comprises of 30-50% w/w bisdemethoxycurcumin, 10-25% w/w demethoxycurcumin and 25-45% w/w curcumin. In yet another related aspect, the total curcuminoids in the composition are in the range of 60-95% w/w. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the proteolytic enzymes are selected from the group consisting of Furin, cathepsin L, and trypsin-like serine proteases. In a further related aspect, the composition is present along with pharmaceutically/nutraceutically accepted excipients, bioavailability enhancers and adjuvants in the form of an extract, powder, emulsion, colloid, or liquid solution.

In another most preferred embodiment, the invention discloses a composition comprising not less than 20% w/w bisdemethoxycurcumin for use in preventing the activation of viral spike proteins by inhibiting the activity of proteolytic enzymes. In a related aspect, the composition comprises of 20-80% w/w bisdemethoxycurcumin. In a further related aspect, the composition comprises of 30-70% bisdemethoxycurcumin. In a further aspect, the composition comprises 40-60% bisdemethoxycurcumin. In a related aspect, the composition further comprises 10-35% w/w demethoxycurcumin and 10-50% w/w curcumin. In a related aspect, the composition further comprises 10-35% w/w demethoxycurcumin and 10-45% w/w curcumin. In another related aspect, the composition comprises of 30-50% w/w bisdemethoxycurcumin, 10-25% w/w demethoxycurcumin and 30-50% w/w curcumin. In another related aspect, the composition comprises of 30-50% w/w bisdemethoxycurcumin, 10-25% w/w demethoxycurcumin and 25-45% w/w curcumin. In yet another related aspect, the total curcuminoids in the composition are in the range of 60-95% w/w. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the proteolytic enzymes are selected from the group consisting of Furin, cathepsin L, and trypsin-like serine proteases. In a further related aspect, the composition is present along with pharmaceutically/nutraceutically accepted excipients, bioavailability enhancers and adjuvants in the form of an extract, powder, emulsion, colloid, or liquid solution.

In another most preferred embodiment, the invention discloses a method of inhibiting the replication of pathogenic viruses in mammalian cells, said method comprising step of bringing into contact mammalian cells infected with the said viruses with a composition comprising not less than 20% w/w bisdemethoxycurcumin, to prevent viral growth and replication. In a related aspect, the composition comprises of 20-80% w/w bisdemethoxycurcumin. In a further related aspect, the composition comprises of 30-70% bisdemethoxycurcumin. In a further aspect, the composition comprises 40-60% bisdemethoxycurcumin. In a related aspect, the composition further comprises 10-35% w/w demethoxycurcumin and 10-50% w/w curcumin. In a related aspect, the composition further comprises 10-35% w/w demethoxycurcumin and 10-45% w/w curcumin. In another related aspect, the composition comprises of 30-50% w/w bisdemethoxycurcumin, 10-25% w/w demethoxycurcumin and 30-50% w/w curcumin. In another related aspect, the composition comprises of 30-50% w/w bisdemethoxycurcumin, 10-25% w/w demethoxycurcumin and 25-45% w/w curcumin. In yet another related aspect, the total curcuminoids in the composition are in the range of 60-95% w/w. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the viral replication is inhibited by preventing the activation of viral spike proteins by inhibiting the activity of proteolytic enzymes. In another related aspect, the mammalian cells are human cells. In a preferred aspect the proteolytic enzymes are selected from the group consisting of Furin, cathepsin L, and trypsin-like serine proteases. In a further related aspect, the composition is present along with pharmaceutically/nutraceutically accepted excipients, bioavailability enhancers and adjuvants in the form of an extract, powder, emulsion, colloid, or liquid solution.

In another most preferred embodiment, the invention discloses a composition comprising not less than 20% w/w bisdemethoxycurcumin for use in inhibiting the replication and growth of pathogenic viruses in mammalian cells. In a related aspect, the composition comprises of 20-80% w/w bisdemethoxycurcumin. In a further related aspect, the composition comprises of 30-70% bisdemethoxycurcumin. In a further aspect, the composition comprises 40-60% bisdemethoxycurcumin. In a related aspect, the composition further comprises 10-35% w/w demethoxycurcumin and 10-50% w/w curcumin. In a related aspect, the composition further comprises 10-35% w/w demethoxycurcumin and 10-45% w/w curcumin. In another related aspect, the composition comprises of 30-50% w/w bisdemethoxycurcumin, 10-25% w/w demethoxycurcumin and 30-50% w/w curcumin. In another related aspect, the composition comprises of 30-50% w/w bisdemethoxycurcumin, 10-25% w/w demethoxycurcumin and 25-45% w/w curcumin. In yet another related aspect, the total curcuminoids in the composition are in the range of 60-95% w/w. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect, the viral replication is inhibited by preventing the activation of viral spike proteins by inhibiting the activity of proteolytic enzymes. In another related aspect, the mammalian cells are human cells. In a preferred aspect the proteolytic enzymes are selected from the group consisting of Furin, cathepsin L, and trypsin-like serine proteases. In a further related aspect, the composition is present along with pharmaceutically/nutraceutically accepted excipients, bioavailability enhancers and adjuvants in the form of an extract, powder, emulsion, colloid, or liquid solution.

In another most preferred embodiment, the invention discloses a method of managing infections caused by viruses in mammals, said method comprising step of administering a composition comprising not less than 20% w/w bisdemethoxycurcumin to mammals infected with said viruses to bring about a reduction in viral load. In a related aspect, the composition comprises of 20-80% w/w bisdemethoxycurcumin. In a further related aspect, the composition comprises of 30-70% bisdemethoxycurcumin. In a further aspect, the composition comprises 40-60% bisdemethoxycurcumin. In a related aspect, the composition further comprises 10-35% w/w demethoxycurcumin and 10-50% w/w curcumin. In a related aspect, the composition further comprises 10-35% w/w demethoxycurcumin and 10-45% w/w curcumin. In another related aspect, the composition comprises of 30-50% w/w bisdemethoxycurcumin, 10-25% w/w demethoxycurcumin and 30-50% w/w curcumin. In another related aspect, the composition comprises of 30-50% w/w bisdemethoxycurcumin, 10-25% w/w demethoxycurcumin and 25-45% w/w curcumin. In yet another related aspect, the total curcuminoids in the composition are in the range of 60-95% w/w. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect the infections caused by viruses are selected from the group consisting of coronavirus disease-19 (COVID-19), acute respiratory failure, pneumonia, acute respiratory distress syndrome, acute liver injury, acute cardiac injury, fugal infections, kidney injury, septic shock, intravascular coagulation, multisystem inflammatory syndrome in children, chronic fatigue, and rhabdomyolysis. In another related aspect, management in infections is brought about by preventing viral replication and inhibiting the activation of viral spike proteins by inhibiting the activity of proteolytic enzymes. In a preferred aspect the proteolytic enzymes are selected from the group consisting of Furin, cathepsin L, and trypsin-like serine proteases. In another related aspect, the mammalian cells are human cells. In another related aspect, the composition is formulated in a composition along with pharmaceutically/nutraceutically acceptable excipients, adjuvants, diluents or carriers and administered orally in the form of tablets, capsules, syrups, gummies, powders, suspensions, emulsions, chewables, candies or eatables.

In another most preferred embodiment, the invention discloses a composition comprising not less than 20% w/w bisdemethoxycurcumin for managing infections caused by viruses in mammals. In a related aspect, the composition comprises of 20-80% w/w bisdemethoxycurcumin. In a further related aspect, the composition comprises of 30-70% bisdemethoxycurcumin. In a further aspect, the composition comprises 40-60% bisdemethoxycurcumin. In a related aspect, the composition further comprises 10-35% w/w demethoxycurcumin and 10-50% w/w curcumin. In a related aspect, the composition further comprises 10-35% w/w demethoxycurcumin and 10-45% w/w curcumin. In another related aspect, the composition comprises of 30-50% w/w bisdemethoxycurcumin, 10-25% w/w demethoxycurcumin and 30-50% w/w curcumin. In another related aspect, the composition comprises of 30-50% w/w bisdemethoxycurcumin, 10-25% w/w demethoxycurcumin and 25-45% w/w curcumin. In yet another related aspect, the total curcuminoids in the composition are in the range of 60-95% w/w. In another related aspect, the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus. In a preferred aspect, the viruses belong to a class of coronavirus. In a further preferred aspect, the virus is SARS-Cov-2. In another related aspect the infections caused by viruses are selected from the group consisting of coronavirus disease-19 (COVID-19), acute respiratory failure, pneumonia, acute respiratory distress syndrome, acute liver injury, acute cardiac injury, fungal infections, kidney injury, septic shock, intravascular coagulation, multisystem inflammatory syndrome in children, chronic fatigue, and rhabdomyolysis. In another related aspect, management in infections is brought about by preventing viral replication and inhibiting the activation of viral spike proteins by inhibiting the activity of proteolytic enzymes. In a preferred aspect the proteolytic enzymes are selected from the group consisting of Furin, cathepsin L, and trypsin-like serine proteases. In another related aspect, the mammalian cells are human cells. In another related aspect, the composition is formulated in a composition along with pharmaceutically/nutraceutically acceptable excipients, adjuvants, diluents or carriers and administered orally in the form of tablets, capsules, syrups, gummies, powders, suspensions, emulsions, chewables, candies or eatables.

The preferred embodiments of the invention are further described in the following illustrative examples

### EXAMPLES

### Example 1: Inhibition of Proteolytic Enzyme (Furin) Activity Materials

The actives present in the composition viz. curcumin, demethoxycurcumin and bisdemethoxycurcumin are isolated and formulated from the rhizomes or spent rhizomes of Curcuma longa into the specific ranges. Alternatively, the actives can also be synthesized chemically and formulated as a composition.

In the present invention the potential of a composition comprising 20-80% w/w bisdemethoxycurcumin, 10-35% w/w demethoxycurcumin and 10-50% w/w curcumin (preferably in the range of 30-50% w/w bisdemethoxycurcumin, 10-25% w/w demethoxycurcumin and 30-50% w/w curcumin or 30-50% w/w bisdemethoxycurcumin, 10-25% w/w demethoxycurcumin and 25-45% w/w curcumin.)—AC3 complex, in inhibiting the enzyme Furin was tested. The specific rage of the composition used in the experiment include 44% w/w bisdemethoxycurcumin, 19% demethoxycurcumin and 38% curcumin. It is to be noted that the range tested is merely illustrative and the results are application to the aforementioned ranges of the composition. The AC3 composition was compared with 95% w/w bisdemethoxycurcumin (BDMC), 95% w/w curcumin and composition enriched in curcumin (95% curcuminoids) which comprises 75-81% curcumin, 15-19% demethoxycurcumin, and 2.2-6.5% bisdemethoxycurcumin—C3 complex.

### Method

Materials: Recombinant human Furin protein, Substrate-p-Glu-Arg-Thr-Lys-Arg-AMC

### Principle

pGlu-Arg-Thr-Lys-Arg-7-amido-4-methylcoumarin was used as a substrate and activity was measured by the fluorescence generated by the release of amino-4-methylcoumarin.

### Protocol

Enzyme (1 μg/ml) was mixed with 25 μM substrate in the presence of different concentrations of test materials. Fluorescence of AMC was recorded at excitation and emission wavelengths of 380 nm and 460 nm respectively in kinetic mode for 20 minutes. Percentage inhibition was calculated using Enzyme and substrate activity as reference.

### Results

The percentage inhibition of the different compounds are mentioned in table 1.

TABLE 1
Percentage inhibition of Furin by different compounds

| | | Percentage Inhibition at concentration | | |
|---|---|---|---|---|
| S. No | Sample | 100 μg/ml | 25 μg/ml | 6.25 μg/ml |
| 1 | 20-80% w/w | 89.48 | 62.30 | 36.37 |
| | bisdemethoxycurcumin, | | | |
| | 10-35% w/w demethoxy- | | | |
| | curcumin and | | | |
| | 10-50% w/w curcumin | | | |
| | (AC3 complex) | | | |
| 2 | Bisdemethoxycurcumin | 78.04 | 61.36 | 33.98 |
| | (BDMC) | | | |
| 3 | Curcumin | 72.64 | 64.15 | 9.64 |
| 4 | 95% curcuminoid | 87.92 | 59.71 | 22.73 |
| | composition | | | |
| | (C3 complex) | | | |

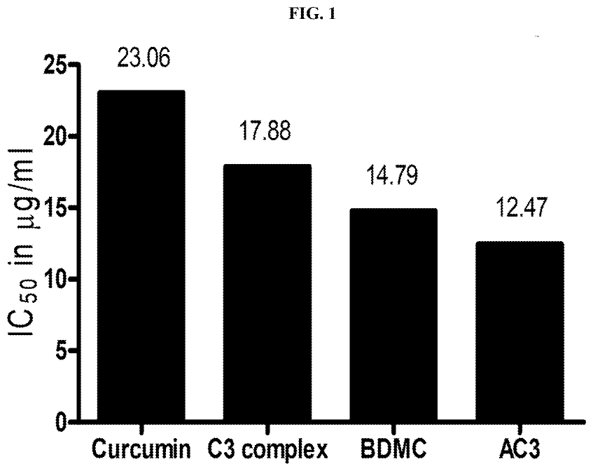

Among the curcuminoid compounds the composition comprising 20-80% w/w bisdemethoxycurcumin, 10-35% w/w demethoxycurcumin and 10-50% w/w curcumin, (termed as AC3 complex showed better inhibition compared to BDMC and curcumin. The IC 50 values are depicted in the FIG. 1.

The AC3 composition showed efficient inhibition of Furin (IC₅₀ 12.47 μg/ml) compared to BDMC (14.79 μg/ml), curcumin (23.06 μg/ml) and curcumin enriched—C3 complex composition (17.88 μg/ml). Thus, viral inhibition studies were performed for the AC3 composition (30-50% w/w bisdemethoxycurcumin, 10-25% w/w demethoxycurcumin and 30-50% w/w curcumin). The IC50 of synthetic inhibitor (positive control) Decanoyl RVKR chloromethyl ketone was 13.41 nM

### Example 2: Anti-Viral Activity in Vero Cells

The antiviral activity of AC3 complex against SARS-CoV-2 was performed in Vero cells at Institute of Life Science, Odisha, India.

Initially, the cytotoxicity of the compound was assessed by MTT assay as per the protocol mentioned in their website (https://www.ils.res.in/biovalidation-service/). Briefly, Vero E6 cells were seeded in the 96 well plate at 80% confluency and treated with various concentrations AC3 complex. For toxicity determination minimum of 3 concentrations were used. MTT assay was performed 48 h post-treatment according to the kit manufacturer's instructions. Each concentration was assayed in triplicates and the percentage cell viability was be calculated with respect to vehicle control.

The results indicated that the maximum non-toxic dose of AC3 complex was found to be 25 μg/ml.

Further, the anti-viral assay was performed according to the protocol mentioned in the validation website (https://www.ils.res.in/biovalidation-service/). Ribavirin (2 μM) was used as positive control in the assay. Vero E6 cells seeded in 96 well plates at 80% confluency were infected with SARS-CoV-2 isolate at an MOI of 0.1 for 2 h. Subsequently the inoculum was aspirated and fresh media containing different concentrations of the test compounds/extract were added to the cells. Each concentration was assayed in triplicates. Compounds showing more the 50% anti-SARS-CoV2 activity was considered for IC₅₀ determination. IC₅₀ was determined using a minimum of 7 different concentrations. 24 h post-infection the supernatant and cells were subjected to viral RNA isolation followed by qRT-PCR for determining the SARS-CoV-2 viral load in the cells (cell associated) and culture supernatants (released virus particles). qRT-PCR was performed using primers specific for the viral spike, nucleocapsid and ORF1a for both released and cell-associated virus. Percentage reduction of viral loads in cells and culture supernatants was plotted in comparison to vehicle treated controls.

The results are tabulated in table 2:

TABLE 2
Anti-viral activity of BDMC composition (AC3 complex)

| | | Concen- | Mean | | % |
|---|---|---|---|---|---|
| | | tration | Ct | Copy | Reduc- |
| Sno | Compound | (μg/ml) | value | number/ml | tion |
| 1 | Infected only | | 22.476 | 297839 | |
| 2 | 20-80% w/w | 5 | 22.693 | 258240 | 13 |
| | bisdemethoxycurcumin, | | | | |
| | 10-35% w/w | | | | |
| | demethoxycurcumin and | | | | |
| | 10-50% w/w curcumin | | | | |
| | AC3 complex | | | | |
| 3 | AC3 complex | 10 | 26.589 | 19936 | 93 |
| 4 | AC3 complex | 15 | 26.915 | 16090 | 94 |
| 5 | AC3 complex | 20 | 29.570 | 2808 | 99 |
| 6 | AC3 complex | 25 | 31.566 | 756 | 99.7 |

The EC₅₀ was evaluated using GraphPad Prisma and was observed to be 6.69 μg/ml. The control Ribavirin was effective in reducing the viral load by 83% compared to infected cells only. Thus, the BDMC enriched composition was more effective than ribavirin and can be effectively used for the managing SARS-CoV-2 related infections.

Evidence indicate that SARS-CoV-2 causes variety of conditions and infections which include acute respiratory failure, pneumonia, acute respiratory distress syndrome, acute liver injury, acute cardiac injury, fugal infections, kidney injury, septic shock, intravascular coagulation, multisystem inflammatory syndrome in children, chronic fatigue, and rhabdomyolysis (WebMD, Complications Coronavirus Can Cause, https://www.webmd.com/lung/coronavirus-complications #1, accessed 2 Sep. 2021). By inhibiting viral load, BDMC enriched AC3 complex will be very effective in managing the above SARS-CoV-2 related infections.

### Example 3: Formulations Containing AC3 Complex

The composition is formulated along with pharmaceutically/nutraceutically acceptable excipients, adjuvants, diluents, stabilizing agents, dispersible gums, bioavailability enhancers or carriers and administered orally in the form of tablets, capsules, syrups, gummies, powders, suspensions, emulsions, chewables, candies or eatables.

In a related aspect the bioavailability enhancer is selected from the group of piperine (BioPerine®), quercetin, garlic extract, ginger extract, and naringin. In another related aspect, the stabilizing agent is selected from the group consisting rosmarinic acid, butylated hydroxyanisole, butylated hydroxytoluene, sodium metabisulfite, propyl gallate, cysteine, ascorbic acid and tocopherols. In yet another related aspect, the dispersible gums are selected from the group consisting of Agar, Alginate, Carrageenan, Gum Arabic, Guar Gum, Locust Bean Gum, Konjac Gum, Xanthan Gum and Pectin.

Tables 3-6 provide illustrative examples of nutraceutical formulations containing bisdemethoxycurcumin

TABLE 3
Tablet

Active Ingredients
AC3 complex (Curcumin BD3 Complex)
Excipients
Microcrystalline cellulose, Colloidal silicon dioxide, Magnesium stearate,
BioPerine ®, Polyvinylpyrrolidone/starch/Hydroxy propyl methyl
cellulose, Hydroxy propyl cellulose

TABLE 4
Capsule

Active Ingredients
ACS complex (Curcumin BD3 Complex)
Excipients
Microcrystalline cellulose, BioPerine ®

TABLE 5
Powder

Active Ingredients
AC3 complex (Curcumin BD3 Complex)
Excipients
BioPerine ®,

TABLE 6
Gummy formulation

Active Ingredients
AC3 complex (Curcumin BD3 Complex)
Excipients
BioPerine ®, Gelatin (270 Bloom Mesh 10), Refined Sugar, Glucose
Corn Syrup, Citric Acid, Lactic Acid, Water, Natural Mango Flavor
M38630, Tartaric Acid, Refined Sugar

TABLE 7
Candy formulation

Active Ingredients
AC3 complex (Curcumin BD3 Complex)
Excipients
BioPerine ®, Sucrose, Liquid Glucose, Flavoring agent, Menthol,
Acidulants (Citric acid/Tartaric Acid/Maleic Acid), Purified water

The above formulations are merely illustrative examples, any formulation containing the above active ingredient intended for the said purpose will be considered equivalent.

Other modifications and variations of the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention and is to be interpreted only in conjunction with the appended claims.

We claim:

1. A method of preventing activation of viral spike proteins by inhibiting the activity of proteolytic enzymes, said method comprising step of bringing into contact proteolytic enzymes with a composition comprising not less than 20% w/w bisdemethoxycurcumin, to bring about an effect of inhibiting the activity of the enzyme.

2. The method as in claim 1, wherein the composition further comprises 10-35% w/w demethoxycurcumin and 10-50% w/w curcumin.

3. The method as in claim 1, wherein the composition comprises of 30-50% w/w bisdemethoxycurcumin, 10-25% w/w demethoxycurcumin and 30-50% w/w curcumin.

4. The method as in claim 1, wherein the composition comprises of 30-50% w/w bisdemethoxycurcumin, 10-25% w/w demethoxycurcumin and 25-45% w/w curcumin.

5. The method as in claim 1, wherein the total curcuminoids in the composition are in the range of 60-95% w/w.

6. The method as in claim 1, wherein the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus.

7. The method as in claim 1, wherein the virus is SARS-Cov-2.

8. The method as in claim 1, wherein the proteolytic enzymes are selected from the group consisting of Furin, cathepsin L, and trypsin-like serine proteases.

9. The method as in claim 1, wherein the composition is present along with pharmaceutically/nutraceutically accepted excipients, bioavailability enhancers and adjuvants in the form of an extract, powder, emulsion, colloid, or liquid solution.

10. A method of inhibiting replication of pathogenic viruses in mammalian cells, said method comprising step of bringing into contact mammalian cells infected with the said viruses with a composition comprising not less than 20% w/w bisdemethoxycurcumin, to prevent viral growth and replication.

11. The method as in claim 10, wherein the composition further comprises 10-35% w/w demethoxycurcumin and 10-45% w/w curcumin.

12. The method as in claim 10, wherein the composition comprises of 30-50% w/w bisdemethoxycurcumin, 10-25% w/w demethoxycurcumin and 30-50% w/w curcumin.

13. The method as in claim 10, wherein the composition comprises of 30-50% w/w bisdemethoxycurcumin, 10-25% w/w demethoxycurcumin and 25-45% w/w curcumin.

14. The method as in claim 10, wherein the total curcuminoids in the composition are in the range of 60-95% w/w.

15. The method as in claim 10, wherein the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus.

16. The method as in claim 10, wherein the virus is SARS-Cov-2.

17. The method as in claim 10, wherein the viral replication is inhibited by preventing the activation of viral spike proteins by inhibiting the activity of proteolytic enzymes.

18. The method as in claim 10, wherein the mammalian cells are human cells.

19. The method as in claim 10, wherein the composition is present along with pharmaceutically/nutraceutically accepted excipients, bioavailability enhancers and adjuvants in the form of an extract, powder, emulsion, colloid, or liquid solution.

20. A method of managing infections caused by viruses in mammals, said method comprising step of administering a composition comprising not less than 20% w/w bisdemethoxycurcumin to mammals infected with said viruses to bring about a reduction in viral load.

21. The method as in claim 20, wherein the composition further comprises 10-35% w/w demethoxycurcumin and 10-45% w/w curcumin.

22. The method as in claim 20, wherein the composition comprises of 30-50% w/w bisdemethoxycurcumin, 10-25% w/w demethoxycurcumin and 30-50% w/w curcumin.

23. The method as in claim 20, wherein the composition comprises of 30-50% w/w bisdemethoxycurcumin, 10-25% w/w demethoxycurcumin and 25-45% w/w curcumin.

24. The method as in claim 20, wherein the total curcuminoids in the composition are in the range of 60-95% w/w.

25. The method as in claim 20, wherein the viruses are selected from the group consisting of Corona Virus, Human immunodeficiency viruses (HIV), Highly pathogenic avian influenza A viruses, Ebola and Marburg viruses, Flaviviruses, Papillomaviruses and Hepatitis B virus.

26. The method as in claim 20, wherein the virus is SARS-Cov-2.

27. The method as in claim 20, wherein the infections caused by viruses are selected from the group consisting of coronavirus disease-19 (COVID-19), acute respiratory failure, pneumonia, acute respiratory distress syndrome, acute liver injury, acute cardiac injury, fungal infections, kidney injury, septic shock, intravascular coagulation, multisystem inflammatory syndrome in children, chronic fatigue, and rhabdomyolysis.

28. The method as in claim 20, wherein the mammal is human.

29. The method as in claim 20, wherein the composition is formulated in a composition along with pharmaceutically/nutraceutically acceptable excipients, adjuvants, diluents or carriers and administered orally in the form of tablets, capsules, syrups, gummies, powders, suspensions, emulsions, chewables, candies or eatables.
